(19) United States
(12) Patent Application Publication
(10) Pub. No.: US 2026/0125652 A1
(43) Pub. Date: May 7, 2026

(54) METHOD FOR EVALUATING NEPHROTOXICITY

(71) Applicants: Kyoto University, Sakyo-ku, Kyoto-shi, Kyoto (JP); Takeda Pharmaceutical Company Limited, Chuo-ku, Osaka-shi, Osaka (JP)

(72) Inventors: Makoto IKEYA, Kyoto (JP); Daisuke KAMIYA, Kyoto (JP); Teppei AKAHOSHI, Kyoto (JP)

(73) Assignees: Kyoto University, Sakyo-ku, Kyoto-shi, Kyoto (JP); Takeda Pharmaceutical Company Limited, Chuo-ku, Osaka-shi, Osaka (JP)

(21) Appl. No.: 19/116,563

(22) PCT Filed: Sep. 28, 2023
(86) PCT No.: PCT/JP2023/035342

§ 371 Date: Mar. 28, 2025

(30) Foreign Application Priority Data
Sep. 29, 2022 (JP) 2022-156017

(51) Int. Cl. C12N 5/071

(52) U.S. Cl. CPC C12N 5/0686; C12N 2501/727; C12N 2503/04; C12N 2506/45

(57) ABSTRACT

The present disclosure provides, as an improved in vitro evaluation system for evaluating nephrotoxicity of a substance such as a compound, a method for evaluating nephrotoxicity of a test substance comprising a step of contacting a test substance with a kidney organoid, wherein the higher cytotoxicity of test substance as compared with a control substance indicates the test substance is likely to have nephrotoxicity.

This application is based upon and claims the benefit of priority from Japanese patent application No. 2022-156017, filed on Sep. 29, 2022, the disclosure of which is incorporated herein in its entirety by reference.

### TECHNICAL FIELD

The present disclosure relates to a method for evaluating nephrotoxicity of a test substance. More particularly, it relates to an in vitro nephrotoxicity evaluation method using a kidney organoid.

### BACKGROUND OF INVENTION

An in vitro evaluation system using a renal proximal tubule epithelial cell line (RPTEC) has been proposed for evaluating nephrotoxicity of a substance such as a compound (see, for example, Non Patent Literature 1). In the in vitro evaluation system using RPTEC, it is difficult to evaluate toxicity to kidney constituent cells (such as a glomerular epithelial cells) except for those of the renal proximal tubule, and therefore, in some cases, a result different from that of a toxic reaction in the living kidney including other cells in addition to those of the renal proximal tubule is obtained.

In relation to the present disclosure, Non Patent Literatures 2 to 4 disclose techniques for inducing a kidney organoid (KiO) from stem cells.

### CITATION LIST

### Non Patent Literature

Non Patent Literature 1:“Nephrotoxicity and Kidney Transport Assessment on 3D Perfused Proximal Tubules”, Marianne K. Vormann, et. al., The AAPS Journal, 2018, 20, Art. No. 90
Non Patent Literature 2:“Kidney organoids from human iPS cells contain multiple lineages and model human nephrogenesis”, Minoru Takasato et. al., Nature, 2015, Volume 526, Page 564-568
Non Patent Literature 3: “Generation of kidney organoids from human pluripotent stem cells”, Minoru Takasato et. al., Nature Protocols, 2016, Volume 11, Page 1681-1692
Non Patent Literature 4:“Plasticity of distal nephron epithelia from human kidney organoids enables the induction of ureteric tip and stalk”, Sara E. Howden et. al., Cell Stem Cell, 2021, Volume 28, Issue 4, 1, Pages 671-684

### SUMMARY OF INVENTION

### Technical Problem

A main object of the present disclosure is to provide an improved in vitro evaluation system for evaluating nephrotoxicity of a substance such as a compound.

### Solution to Problem

In order to solve the problem, the present disclosure provides the following [1] to [32]:
[1] A method for evaluating nephrotoxicity of a test substance comprising a step of contacting a test substance with a kidney organoid, wherein the higher cytotoxicity of test substance as compared with a control substance indicates the test substance is likely to have nephrotoxicity.
[2] A method for screening a test substance likely to have nephrotoxicity comprising the steps of: (1) contacting a test substance with a kidney organoid; and (2) selecting a test substance showing higher cytotoxicity as compared with a control substance.
[3] A method for screening a test substance likely to have no nephrotoxicity or likely to have low nephrotoxicity comprising the steps of (1) contacting a test substance with a kidney organoid; and (2) selecting a test substance showing no cytotoxicity or lower cytotoxicity as compared with a control substance.
[4] The method according to any one of [1] to [3], wherein the kidney organoid is derived from a human induced pluripotent stem cell.
[5] The method according to any one of [1] to [4], wherein the kidney organoid is enriched in glomerular epithelial cell-like cells.
[6] The method according to [5], wherein 30 to 80% of cells contained in the kidney organoid are glomerular epithelial cell-like cells.
[7] The method according to [5] or [6] further comprising a step (a) of culturing a pluripotent stem cell in the presence of a 9.0 to 10.5 μM GSK3β inhibitor to obtain the kidney organoid enriched in glomerular epithelial cell-like cells.
[7a] The method according to [7] comprising the step (a) before the step (1).
[8] The method according to [7] or [7a], wherein the GSK3β inhibitor is CHIR99021 (6-[[2-[[4-(2,4-dichlorophenyl) -5-(4-methyl-1H-imidazol-2-yl)2-pyrimidinyl]amino]ethyl]amino]nicotinonitrile).
[9] The method according to any one of [1] to [4], wherein the kidney organoid is enriched in renal proximal tubule epithelial cell-like cells.
[10] The method according to [9], wherein 5 to 30% of cells contained in the kidney organoid are renal proximal tubule epithelial cell-like cells.
[11] The method according to [9] or [10] further comprising a step (b) of culturing a pluripotent stem cell in the presence of a 5.5 to 8.5 μM GSK3β inhibitor to obtain the kidney organoid enriched in renal proximal tubule epithelial cell-like cells.
[12] The method according to [11], wherein the GSK3β inhibitor is CHIR99021(6 -[[2-[[4-(2,4-dichlorophenyl) -5-(4-methyl-1H-imidazol-2-yl)2-pyrimidinyl]amino]ethyl]amino]nicotinonitrile).
[13 ] A method for predicting a site in the kidney to which a test substance shows toxicity comprising the steps of: (A) contacting a test substance with a kidney organoid enriched in glomerular epithelial cell-like cells; (B) contacting the test substance with a kidney organoid enriched in renal proximal tubule epithelial cell-like cells; and (C) comparing cytotoxicity obtained in the step (A) and cytotoxicity obtained in the step (B) to predict that the site showing toxicity is the glomerulus when the cytotoxicity obtained in the step (A) is higher, and that the site showing toxicity is the renal proximal tubule when the cytotoxicity obtained in the step (B) is higher.
[14] The method according to [13], wherein 30 to 80% of cells contained in the kidney organoid enriched in glomerular epithelial cell-like cells are glomerular epithelial cell-like cells, and 5 to 30% of cells contained in the kidney organoid enriched in renal proximal tubule epithelial cell-like cells are renal proximal tubule epithelial cell-like cells.
[15] The method according to [13] or [14] further comprising the steps of (a) culturing a pluripotent stem cell in the presence of a 9.0 to 10.5 μM GSK3β inhibitor to obtain the kidney organoid enriched in glomerular epithelial cell-like cells; and (b) culturing a pluripotent stem cell in the presence of a 5.5 to 8.5 μM GSK3β inhibitor to obtain the kidney organoid enriched in renal proximal tubule epithelial cell-like cells.
[16] The method according to [15], wherein the GSK3β inhibitor is CHIR99021(6 -[[2-[[4-(2,4-dichlorophenyl) -5-(4-methyl-1H-imidazol-2-yl)2-pyrimidinyl]amino]ethyl]amino]nicotinonitrile).
[17] A method for screening nephrotoxicity of a drug candidate compound comprising the steps of (1) contacting a test substance with a kidney organoid; and (2) selecting, as a drug candidate compound, a test substance showing no cytotoxicity or lower cytotoxicity as compared with a control substance.
[18] The method according to [17], wherein the kidney organoid is derived from a human induced pluripotent stem cell.
[19] The method according to [17] or [18], wherein the kidney organoid is enriched in glomerular epithelial cell-like cells.
[20] The method according to [19], wherein 30 to 80% of cells contained in the kidney organoid are glomerular epithelial cell-like cells.
[21] The method according to [19] or [20] further comprising a step (a) of culturing a pluripotent stem cell in the presence of a 9.0 to 10.5 μM GSK3β inhibitor to obtain the kidney organoid enriched in glomerular epithelial cell-like cells.
[22] The method according to [21], wherein the GSK3β inhibitor is CHIR99021(6 -[[2-[[4-(2,4-dichlorophenyl) -5-(4-methyl-1H-imidazol-2-yl)2-pyrimidinyl]amino]ethyl]amino]nicotinonitrile).
[23] The method according to [17] or [18], wherein the kidney organoid is enriched in renal proximal tubule epithelial cell-like cells.
[24] The method according to [23], wherein 5 to 30% of cells contained in the kidney organoid are renal proximal tubule epithelial cell-like cells.
[25] The method according to [23] or [24] further comprising a step (b) of culturing a pluripotent stem cell in the presence of a 5.5 to 8.5 μM GSK3β inhibitor to obtain the kidney organoid enriched in renal proximal tubule epithelial cell-like cells.
[26] The method according to [25], wherein the GSK3β inhibitor is CHIR99021(6 -[[2-[[4-(2,4-dichlorophenyl) -5-(4-methyl-1H-imidazol-2-yl)2-pyrimidinyl]amino]ethyl] amino]nicotinonitrile).
[27] A kit for evaluating nephrotoxicity of a test substance comprising a kidney organoid.
[28] The kit according to [27], wherein the kidney organoid is enriched in glomerular epithelial cell-like cells.
[29] The kit according to [27], wherein the kidney organoid is enriched in renal proximal tubule epithelial cell-like cells.
[30] A method for producing a kidney organoid enriched in glomerular epithelial cell-like cells comprising a step (a) of culturing a pluripotent stem cell in the presence of a 9.0 to 10.5 μM GSK3β inhibitor.
[31] A method for producing a kidney organoid enriched in renal proximal tubule epithelial cell-like cells comprising a step (b) of culturing a pluripotent stem cell in the presence of a 5.5 to 8.5 μM GSK3β inhibitor.
[32] The production method according to [30] or [31], wherein the GSK3β inhibitor is CHIR99021 (6-[[2-[[4-(2,4-dichlorophenyl) -5-(4-methyl-1H-imidazol-2-yl)2-pyrimidinyl]amino]ethyl]amino]nicotinonitrile).

### DEFINITIONS

The term “kidney organoid” means a three-dimensional structure constituting a kidney tissue of a living body, and containing at least one or more cell populations.

The term “glomerular epithelial cell-like cell” means a cell induced from a stem cell (such as a pluripotent stem cell), and having the same properties as a glomerular epithelial cell. The phrase “the same properties as a glomerular epithelial cell” refers to being positive to specific staining against a glomerular epithelial cell, and means that a glomerular epithelial cell marker gene is expressed therein. A glomerular epithelial cell is CCND1, CDH6, EMX2, and SOX4 positive.

The term “renal proximal tubule epithelial cell-like cell” means a cell induced from a stem cell (such as a pluripotent stem cell), and having the same properties as a renal proximal tubule epithelial cell. The phrase “the same properties as a renal proximal tubule epithelial cell” refers to being positive to specific staining against a renal proximal tubule epithelial cell, and means that a renal proximal tubule epithelial cell marker gene is expressed therein. A renal proximal tubule epithelial cell is LTL, DAB2, CUBN, and SLC34A1 positive.

The term “intermediate mesoderm” refers to a type of embryo generated from the mesoderm in the ontogeny, and is a cell capable of differentiating into the pronephros, the mesonephros, the mesonephric duct, the metanephros, the adrenal cortex, and the gonad. An intermediate mesoderm is OSR 1 (odd-skipped related 1) positive.

The term “GSK3β inhibitor” refers to a substance having inhibitory activity against GSK3β (glycogen synthase kinase 3β). GSK3 (glycogen synthase kinase 3) is a type of serine/threonine protein kinase, and is involved in a large number of signaling pathways related to production of glycogen, apoptosis, and maintenance of stem cells. GSK3 has two isoforms of GSK3α and GSK3β. The GSK3β inhibitor is not especially limited as long as it has GSK3β inhibitory activity, and may be a substance having GSK3α inhibitory activity in addition to GSK3β inhibitory activity.

The term “culture” refers to maintaining, proliferating (growing), and/or differentiating cells in an in vitro environment. The term “culturing” means maintaining, proliferating (growing), and/or differentiating cells outside a tissue or body, for example, in a cell culture dish or flask.

The term “pluripotency” means an ability to differentiate into tissues and cells having various different forms and functions, and to differentiate into cells of any system of the three germ layers. The “pluripotency” does not include an ability to differentiate into the blastodisc, and therefore does not have an ability to develop an individual, and thus, is distinguished from “totipotency” of the ability to differentiate into all the tissues of a living body including the blastodisc.

The term “multipotency” means an ability to differentiate into cells of a plurality of limited numbers of systems. For example, a mesenchymal stem cell, a hematopoietic stem cell, and a neural stem cell are multipotent but not pluripotent.

The term “marker” refers to a “marker protein” or a “marker gene”, and means a protein or a gene thereof specifically expressed on the cell surface, in the cytoplasm, and/or in the nucleus of a prescribed cell type. The marker may be a positive selection marker or a negative selection marker. Preferably, the marker is a cell surface marker, and particularly when a cell surface positive selection marker is used, living cells can be concentrated, isolated and/or detected.

A marker protein can be detected by immunoassay using an antibody specific to the marker protein, such as ELISA, immunostaining, and flowcytometry. As an antibody specific to a marker protein, an antibody binding to a specific amino acid sequence of the marker protein, or to a specific sugar chain or the like bound to the marker protein can be used. When a marker protein is expressed in a cell but is not expressed on the cell surface (for example, a transcription factor, or a subunit thereof), the target marker protein can be detected by expressing a reporter protein together with the marker protein, and detecting the reporter protein (for example, Non Patent Literature 4). This method is preferably employed when an appropriate cell surface marker is not found. The detection of a marker gene can be performed by nucleic acid amplification methods and/or nucleic acid detection methods known in this field, such as RT-PCR, microarray, biochip, and RNAseq methods.

The term “expression” is defined as transcription and/or translation of a specific nucleotide sequence driven by an intracellular promoter.

The term “being positive” or “being expressed” means that a protein or a gene is expressed at a level detectable by a method known in the field. The detection of a protein can be performed by immunoassay using an antibody, such as ELISA, immunostaining, or flowcytometry. When a protein is expressed in a cell but is not expressed on the cell surface (for example, a transcription factor, or a subunit thereof), the target protein can be detected by expressing a reporter protein together with the target protein, and detecting the reporter protein. The detection of a gene can be performed by a nucleic acid amplification method and/or a nucleic acid detection method, such as RT-PCR, microarray, biochip, and RNAseq methods.

The term “being negative” or “being not expressed” means that the expression level of the protein or the gene is lower than a detection lower limit in all or any one of the known methods described above. The detection lower limit of the expression of the protein or the gene may be different among the methods.

The term “comprise(s)” or “comprising” means inclusion of, but not limited to, an element following the term. Accordingly, inclusion of an element following the term is suggested, but exclusion of another optional element is not suggested.

### Advantageous Effects of Invention

The present disclosure provides an improved in vitro evaluation system for evaluating nephrotoxicity of a substance such as a compound.

### BRIEF DESCRIPTION OF DRAWINGS

FIG. 1 illustrates an experimental scheme of steps 2-3 and 3 in the outline of differentiation induction from iPSC to KiO.

FIG. 2 illustrates CHIR addition period conditions (1) to (3) in Example 1.

FIG. 3 illustrates fluorescence microscope images of KiOs induced with CHIR addition period changed (Example 1).

FIG. 4 illustrates fluorescence microscope images of KiOs induced with the addition concentration of CHIR changed (Example 1).

FIG. 5 illustrates phase-contrast microscope images of cells having a renal tubule-like shape induced in KiOs (Example 1).

FIG. 6 illustrates patterns of gene expression of KiOs induced at CHIR addition concentrations of 6 μM, 8 μM, and 10 μM.

FIG. 7 illustrates evaluation results of cytotoxicity of Cyclosporin A obtained by using a KiO prepared in accordance with a method described in Example 2 and a renal proximal tubule epithelial cell line (RPTEC) (Example 3).

FIG. 8 illustrates results of evaluation, based on the intracellular ATP amount, of cytotoxicity of Cyclosporin A obtained by using a KiO having a high ratio of renal proximal tubule epithelial cell-like cells produced in accordance with a method described in Example 1 (Tubule rich), a KiO having a high ratio of glomerular epithelial cell-like cells (Glomerulus rich), and a KiO having an intermediate ratio between renal proximal tubule epithelial cell-like cells and glomerular epithelial cell-like cells (Intermediate) (Example 3).

FIG. 9 illustrates results of evaluation, based on the LDH leakage amount, of cytotoxicity of Cyclosporin A obtained by using a KiO having a high ratio of renal proximal tubule epithelial cell-like cells produced in accordance with the method described in Example 1 (Tubule rich), a KiO having a high ratio of glomerular epithelial cell-like cells (Glomerulus rich), and a KiO having an intermediate ratio between renal proximal tubule epithelial cell-like cells and glomerular epithelial cell-like cells (Intermediate) (Example 3).

FIG. 10 illustrates results of evaluation, based on the amount of secreted KIM-1, of cytotoxicity of Cyclosporin A obtained by using a KiO having a high ratio of renal proximal tubule epithelial cell-like cells produced in accordance with the method described in Example 1 (Tubule rich), a KiO having a high ratio of glomerular epithelial cell-like cells (Glomerulus rich), and a KiO having an intermediate ratio between renal proximal tubule epithelial cell-like cells and glomerular epithelial cell-like cells (Intermediate) (Example 3).

### DESCRIPTION OF EMBODIMENTS

Now, a preferred embodiment for implementing the present disclosure will be described. It is noted that the embodiment described below is merely an example of representative embodiments of the present disclosure, by which the scope of the present disclosure should not be narrowly interpreted.

### 1. Method for Evaluating Nephrotoxicity of Test Substance

A method for evaluating nephrotoxicity of a test substance of the present disclosure includes a step of contacting a test substance with a kidney organoid, and determines that the test substance is likely to have nephrotoxicity when the test substance shows higher cytotoxicity as compared with a control substance.

### Test Substance

The test substance is not especially limited, and may be a compound such as an organic low molecular weight compound; a metal complex; a polymer (including an aptamer) such as a peptide, a protein, an antibody, or a nucleic acid; a sugar; a lipid; a tissue extract; a cell extract; a cell culture supernatant; a plant extract; a microorganism product; or a biological material such as blood or urine, and particularly, may be a drug candidate compound. The test substance may be a synthetic compound, or a compound extracted from a natural product.

### Pluripotent Stem Cell

The kidney organoid may be induced from a stem cell in vitro. Induction of a kidney organoid from a pluripotent stem cell can be performed in accordance with a conventionally known method (for example, those described in Non Patent Literatures 2 to 4).

The term “pluripotent stem cell” refers to a stem cell that is capable of differentiating into tissues or cells having various different biological forms and functions, and has an ability to differentiate into cells of any system of the three germ layers (endoderm, mesoderm, and ectoderm). Examples of the pluripotent stem cell include an embryonic stem cell (ESC), an embryonic stem cell derived from a cloned embryo obtained through nuclear transfer, a spermatogonial stem cell, an embryonic germ cell, and an induced pluripotent stem cell (herein, also referred to as an “iPSC”).

The term “multipotent stem cell” refers to a stem cell having an ability to differentiate into cells of a plurality of limited numbers of systems. Examples of the multipotent stem cell include a dental pulp stem cell, an oral mucosa-derived stem cell, a hair follicle stem cell, and a somatic stem cell derived from a cultured fibroblast or a bone marrow stem cell.

The pluripotent stem cell is preferably an ESC or an iPSC.

As the “ESC”, specifically as a mouse ESC, various mouse ESC lines established by inGenious Targeting Laboratory Inc., RIKEN (Institute of Physical and Chemical Research) and the like can be used, and as a human ESC, various human ESC lines established by Wisconsin University, NIH, RIKEN, Kyoto University, National Center for Child Health and Development, Cellartis and the like can be used. For example, as a human ESC line, CHB-1 to CHB-12, RUES1, RUES2, and HUES1 to HUES28 distributed by ESI Bio, H1 and H9 distributed by WiCell Research Institute Inc., and KhES-1, KhES-2, KhES-3, KhES-4, KhES-5, SSES1, SSES2, and SSES3 distributed by RIKEN may be used.

The term “iPSC” refers to a cell obtained by reprogramming with a specific factor (nuclear reprogramming factor) introduced into a mammal somatic cell or an undifferentiated stem cell. There are currently various iPSCs, and not only an iPSC established by Yamanaka et al. by introducing four factors of Oct3/4, Sox2, Klf4, and c-Myc into a mouse fibroblast (Takahashi K., Yamanaka S., Cell (2006) 126: 663-676) but also a human cell-derived iPSC established by introducing the same four factors into a human fibroblast (Takahashi K., Yamanaka S., et al., Cell (2007) 131: 861-872), a Nanog-iPSC established with expression of Nanog used as an indicator after introducing the four factors (Okita, K., Ichisaka, T., and Yamanaka S. (2007), Nature 448, 313-317), an iPSC produced by a method not containing c-Myc (Nakagawa M., Yamanaka S., et al., Nature Biotechnology (2008) 26, 101-106), and an iPSC established by introducing six factors by a virus-free method (Okita K. et al., Nat. Methods 2011 May; 8(5): 409-12, Okita K. et a., Stem Cells 31(3): 458-66) can be used. Besides, an induced pluripotent stem cell established by introducing four factors of OCT3/4, SOX2, NANOG, LIN28 produced by Thomson et al., (Yu J., Thomson JA. et al., Science (2007) 318: 1917-1920), an induced pluripotent stem cell produced by Daley et al., (Park IH, Daley GQ. et al., Nature (2007) 451: 141-146), an induced pluripotent stem cell produced by Sakurada et al., (Japanese Patent Laid-Open No. 2008-307007) and the like can be used.

In addition, any one of induced pluripotent stem cells known in this field described in all published theses (such as Shi Y., Ding S., et al., Cell Stem Cell, (2008) Vol3, Issue 5, 568-574; Kim JB., Scholer HR., et al., Nature, (2008) 454, 646-650; Huangfu D., Melton, DA., et al., Nature Biotechnology, (2008) 26, No 7, 795-797) or patents (such as Japanese Patent Laid-Open No. 2008-307007, Japanese Patent Laid-Open No. 2008-283972, US2008-2336610, US2009-047263, WO2007-069666, WO2008-118220, WO2008-124133, WO2008-151058, WO2009-006930, WO2009-006997, WO2009-007852) can be used.

As the induced pluripotent stem cell line, various iPSC lines established by NIH, RIKEN, Kyoto University and the like are usable. Examples of a human iPSC line include HiPS-RIKEN-1A, HiPS-RIKEN-2A, HiPS-RIKEN-12A, and Nips-B2 of RIKEN, and 253G1, 201B7, 409B2, 454E2, 606A1, 610B1, 648A1, and 1231A3 of Kyoto University, among which 1231A1 and 1231A3 are preferred, and 1231A3 is more preferred.

### Induction of Kidney Organoid From Pluripotent Stem Cell

The induction of a kidney organoid from a pluripotent stem cell may be, specifically when a human iPSC is used, performed as follows.

First, human iPSCs are cultured in a medium containing a GSK3β inhibitor, and then cultured in a medium containing FGF9 and heparin, the resultant is dissociated into single cells, and about 1×10⁴ to 1×10⁵ cells are further cultured in a medium containing FGF9 and heparin to form a cell aggregate (intermediate mesoderm spheroid) (step i).

Subsequently, the intermediate mesoderm spheroid is subjected to air-liquid interface culture with a medium containing the GSK3β inhibitor successively changed to a medium containing FGF9 and heparin, thereby obtaining a kidney organoid (step ii).

The concentration of the GSK3β inhibitor in the step i is, for example, 0.1 to 30 μM, preferably 1 to 20 μM, more preferably 6 to 10 μM, and particularly preferably 8 μM.

The concentration of FGF9 is, for example, 0.1 ng/ml to 1 μg/ml, preferably 1 to 500 ng/ml, more preferably 10 to 300 ng/ml, and particularly preferably about 200 ng/ml.

The concentration of heparin is, for example, 0.01 to 100 μg/ml, preferably 0.1 to 10 μg/ml, and particularly preferably about 1 μg/ml.

The culture period in the medium containing the GSK3β inhibitor is, for example, 3 to 5 days, and particularly 4 days.

The culture period in the medium containing FGF9 and heparin is, for example, 1 to 3 days, and particularly 1 day before the single-cell dissociation, and is, for example, 0.5 to 2 days, and particularly 2 days after the single-cell dissociation.

The concentration of the GSK3β inhibitor in the step ii is, for example, 0.1 to 30 μM, preferably 1 to 10 μM, more preferably 3 to 7 μM, and particularly preferably 5 μM.

The concentration of FGF9 is, for example, 0.1 ng/ml to 1 μg/ml, preferably 1 to 500 ng/ml, more preferably 10 to 300 ng/ml, and particularly preferably about 200 ng/ml.

The concentration of heparin is, for example, 0.01 to 100 μg/ml, preferably 0.1 to 10 μg/ml, and particularly preferably about 1 μg/ml.

The culture period in the medium containing the GSK3β inhibitor is, for example, 0.5 to 2 hours, and particularly 1 hour.

The culture period in the medium containing FGF9 and heparin is, for example, 0.5 to 2 days, and particularly 1 day.

Subsequently to the step ii, a step of culturing the kidney organoid in a medium containing FGF9 and heparin, and then in a medium containing heparin may be performed.

The culture period in the medium containing FGF9 and heparin is, for example, 3 to 7 days, and particularly 4 days.

The culture period in the medium containing heparin is, for example, 7 to 25 days, and particularly 14 days.

The concentrations of FGF9 and heparin may be the same as those employed in the step ii.

For confirming the production of the intermediate mesoderm and the kidney organoid, for example, a method for measuring expression of a marker protein or a marker gene may be employed.

When OSR1 is expressed in the obtained cell aggregate, the cell aggregate can be determined as an intermediate mesoderm.

When the obtained cell aggregate has EPO-producing ability under low oxygen condition, and markers for the renal interstitial cells, the glomerulus, and the renal proximal tubule are expressed therein, the cell aggregate can be determined as a kidney organoid. Examples of a renal interstitial cells marker include FOXD1, PDGFRβ, and CD73. Examples of a glomerulus marker include WT1 and NPHS1. Examples of a renal proximal tubule marker include LTL, CUBN, and E-cadherin.

Besides, the production of the intermediate mesoderm may be confirmed also by, for example, confirming differentiation-inducing ability of the cell into the glomerulus or the renal proximal tubule.

### GSK3β Inhibitor

Examples of the GSK3β inhibitor include CHIR98014 (N6-[2-[[4-(2,4-dichlorophenyl)-5-(1H-imidazol-2-yl)-2-pyrimidinyl]amino]ethyl]-3-nitro-2, 6-pyridinediamine), CHIR99021(6-{2-[4-(2,4-Dichloro-phenyl)-5-(5-methyl-1H-imidazol-2-yl) -pyrimidin-2-ylamino]-ethylamino}-nicotinonitrile), CP21R7 (CP21R7), LY2090314 (3-[9-Fluoro-1, 2,3,4-tetrahydro-2-(1-piperidinylcarbonyl) pyrrolo[3,2,1-jk] [1,4]benzodiazepin-7-yl]-4-imidazo[1,2-a]pyridin-3-yl-1h-pyrrole-2,5-dione), TDZD-8(2-methyl-4-(phenylmethyl)-1,2,4-thiadiazolidine-3, 5-dione), SB216763 (3-(2,4-Dichlorophenyl)-4-(1-methyl-1H-indol-3-yl)-1H-pyrrole-2, 5-dione), TWS-119(3-[[6-(3-Aminophenyl) -7H-pyrrolo[2,3-d]pyrimidin-4-yl]oxy]phenol), Kenpaullone, 1-Azakenpaullone, SB415286 ([3-[(3-Chloro-4-hydroxyphenyl) amino]-4-(2-nitrophenyl)-1Hpyrrole-2,5-dione]) and AR-AO144-18 (1-[(4-methoxyphenyl)methyl]-3-(5-nitro-1, 3-thiazol-2-yl)urea), CT20026, BIO ((2′Z, 3′E) 6-bromoindirubin-3′-oxime), BIO-acetoxime, a pyridocarbazole-cyclopentadienylruthenium complex, OTDZT, alpha-4-dibromoacetophenone, and lithium. A combination of two or more of these may be used.

The GSK3β inhibitor is not limited to these, but an antisense oligonucleotide or siRNA against an mRNA of GSK3β, an antibody binding to GSK3β, a dominant negative GSK3β mutant and the like may be used as the GSK3β inhibitor, and these are commercially available, or may be synthesized in accordance with any of known methods.

### Medium

A basal medium is not especially limited, and for example, STEMdiff APEL2 medium (STEMCELL Technologies, ST-05275), TeSR1 medium, and Chemically Defined Medium (CDM) are suitably used. In addition, BME medium, BGJb medium, CMRL 1066 medium, Glasgow MEM medium, Improved MEM (IMEM) medium, Improved MDM (IMDM) medium, Medium 199 medium, Eagle MEM medium, αMEM medium, DMEM medium (High glucose, Low glucose), DMEM/F12 medium, Ham's medium, RPMI 1640 medium, Fischer's medium, and a mixed medium of these may be used.

The CDM medium is not especially limited, and for example, a medium prepared from Iscove's modified Dulbecco's medium (manufactured by GE Healthcare) may be used.

The basal medium may be supplemented with a substance usually used in cell culture, such as Ham's F-12 nutrient mixture, albumins including human serum albumin, polyvinyl alcohol (PVA), Deionized BSA, linoleic acid, linolenic acid, cholesterol, insulin, apotransferrin, selenium, ethanolamine, monothioglycerol, Protein-free hybridoma mixture II (PFHMII), ascorbic acid, L-alanyl-L-glutamine, and/or an antibiotic.

### Ratio Between Glomerular Epithelial Cell-Like Cells and Renal Proximal Tubule Epithelial Cell-Like Cells

When the addition period and the concentration of the GSK3β inhibitor in the step i are changed, kidney organoids having different ratios between the glomerular epithelial cell-like cells and the renal proximal tubule epithelial cell-like cells can be obtained.

Specifically, when a kidney organoid enriched in and having a high ratio of the glomerular epithelial cell-like cells is to be induced, the culture is performed in a basal medium supplemented with a 9.0 to 10.5 μM (preferably 9.5 to 10.5 μM) GSK3β inhibitor for 4 days, or in a basal medium supplemented with a 7 to 9 μM (preferably 7.5 to 8.5 μM) GSK3β inhibitor for 5 days.

When a kidney organoid enriched in and having a high ratio of the renal proximal tubule epithelial cell-like cells is to be induced, the culture is performed in a basal medium supplemented with a 5.5 to 7.0 μM (preferably 5.5 to 6.5 μM) GSK3β inhibitor for 4 days, or in a basal medium supplemented with a 6.5 to 8.5 μM (preferably 7.5 to 8.5 μM) GSK3β inhibitor for 3 days.

When the culture is performed, in the step i, in a basal medium supplemented with the GSK3β inhibitor in a concentration of more than 7.0 μM and less than 9.0 μM for 4 days, a kidney organoid containing the glomerular epithelial cell-like cells and the renal proximal tubule epithelial cell-like cells in equivalent amounts can be induced.

When an intermediate condition between the condition of the addition period and the concentration of the GSK3β inhibitor for inducing a kidney organoid enriched in the glomerular epithelial cell-like cells and the condition of the addition period and the concentration of the GSK3β inhibitor for inducing a kidney organoid enriched in the renal proximal tubule epithelial cell-like cells is employed, a kidney organoid having an intermediate ratio between the renal proximal tubule epithelial cell-like cells and the glomerular epithelial cell-like cells is obtained.

In the rest of the period in the step i after completing the addition of the GSK3β inhibitor, the culture is performed in a medium containing FGF9 and heparin.

In a kidney organoid enriched in the glomerular epithelial cell-like cells, 30 to 80% of cells contained in the kidney organoid can be the glomerular epithelial cell-like cells.

In a kidney organoid enriched in the renal proximal tubule epithelial cell-like cells, 5 to 30% of cells contained in the kidney organoid can be the renal proximal tubule epithelial cell-like cells.

### Contacting Step

The step of contacting a test substance with a kidney organoid may be performed by adding the test substance to a medium in which the kidney organoid is maintained in an appropriate container such as a dish, a flask, or a microplate. The step of contacting a test substance with a kidney organoid can be performed by, for example, introducing a basal medium containing the test substance into an air phase and/or liquid phase of a transwell plate in which the kidney organoid is prepared on an air-liquid interface.

### Measurement of Cytotoxicity

The cytotoxicity of the kidney organoid having contacted with the test substance can be measured and evaluated in accordance with any of conventionally known methods. Known methods are, for example, methods using, as an indicator of the cytotoxicity, decrease of the intracellular ATP amount, increase of the amount of leakage of an intracellular enzyme to the outside of the cell, or increase of the amount of a marker protein secreted into the medium.

### Determination of Nephrotoxicity

It can be determined that a test substance showing cytotoxicity to the kidney organoid is likely to have nephrotoxicity.

More preferably, it is determined that a test substance showing higher cytotoxicity as compared with a control substance known to have no or low nephrotoxicity is likely to have nephrotoxicity.

The determination criterion may be appropriately set in accordance with the purpose of the evaluation, and the type of the substance to be evaluated, and for example, a substance is determined to have cytotoxicity when the indicator is higher, than that of the control substance, by 10%, 20%, 30%, 40%, 50%, 60%, 70%, 80%, 90%, or 100%, and preferably by 100%, 200%, 300%, 400%, 500%, 600%, 700%, 800%, 900%, or 1000% or more.

In the method for evaluating nephrotoxicity of a test substance of the present disclosure, differently from an in vitro evaluation method using a renal proximal tubule epithelial cell line (RPTEC), a kidney organoid containing both the glomerular epithelial cell-like cells and the renal proximal tubule epithelial cell-like cells is used, and therefore, the toxicity in vivo of the test substance may be more appropriately reflected on the evaluation.

Besides, in the method for evaluating nephrotoxicity of a test substance of the present disclosure, when kidney organoids respectively having different ratios between the glomerular epithelial cell-like cells and the renal proximal tubule epithelial cell-like cells are used, it may be evaluated which of the glomerulus and the renal proximal tubule the test substance shows higher toxicity to.

### 2. Method for Screening Test Substance on Nephrotoxicity

The method for evaluating nephrotoxicity of a test substance of the present disclosure can be used in a method for screening a test substance likely to have nephrotoxicity, or a test substance likely to have no or low nephrotoxicity.

A plurality of test substances are subjected to evaluation for cytotoxicity by the nephrotoxicity evaluation method described above, and a test substance showing cytotoxicity to the kidney organoid (more preferably, a test substance showing higher cytotoxicity as compared with a control substance) is selected as a substance likely to have nephrotoxicity.

Alternatively, a plurality of test substances are subjected to evaluation for cytotoxicity by the nephrotoxicity evaluation method described above, and a test substance showing no cytotoxicity to the kidney organoid, or a test substance showing lower cytotoxicity as compared with a control substance is selected as a substance likely to have no or low nephrotoxicity. Such screening can be used for screening for nephrotoxicity of a drug candidate compound.

Besides, when kidney organoids having different ratios between the glomerular epithelial cell-like cells and the renal proximal tubule epithelial cell-like cells are used, a screening system focusing toxicity to one of the glomerulus and the renal proximal tubule can be obtained.

### 3. Method for Predicting Site in Kidney to Which Test Substance Shows Toxicity

The method for evaluating nephrotoxicity of a test substance of the present disclosure can be used also in a method for predicting a cite in the kidney to which a test substance shows toxicity.

Steps (A) of contacting a test substance with a kidney organoid enriched in the glomerular epithelial cell-like cells, and (B) of contacting the test substance with a kidney organoid enriched in the renal proximal tubule epithelial cell-like cells are performed.

Then, in a step (C), cytotoxicity obtained in the step (A) and cytotoxicity obtained in the step (B) are compared, and it can be predict that a site showing toxicity is the glomerulus when the cytotoxicity obtained in the step (A) is higher, and that the site showing toxicity is the renal proximal tubule when the cytotoxicity obtained in the step (B) is higher.

### 4. Kit

The present disclosure also provides a kit comprising a kidney organoid to be used for the method for evaluating nephrotoxicity of a test substance, the method for screening a test substance for nephrotoxicity, and the method for predicting a site in the kidney to which a test substance shows toxicity.

The kit of the present disclosure may include the kidney organoid enriched in the glomerular epithelial cell-like cells, or the kidney organoid enriched in the renal proximal tubule epithelial cell-like cells.

The kit of the present disclosure may comprise, in addition to the kidney organoid, media and reagents (such as a GSK3β inhibitor, FGF, and heparin) used for culturing the kidney organoid, and a reagent for measuring cytotoxicity to the kidney organoid.

The kit of the present disclosure may include, instead of the kidney organoid, a pluripotent stem cell, and media and reagents (such as a GSK3β inhibitor, FGF, and heparin) used for differentiation-inducing the cell into a kidney organoid.

### EXAMPLES

### Differentiation Induction of Induced Pluripotent Stem Cell (iPSC)-Derived Kidney Organoid (KiO) (Outline)

(1) Differentiation Induction From iPSC Into Intermediate Mesoderm Spheroid
Step 1 (Day 1): iPSC Seeding

1231A3 iPSCs were seeded in a laminin coated (iMatrix-511 silk: Nippi Inc.) 6-well plate at 5.76×10⁵ cells/well. The resultant was cultured overnight in an incubator at 37° C. and 5% CO₂.

As a medium, AK03N (Ajinomoto Co., Inc.) supplemented with 10 μM Y 27632 was used in an amount of 2 ml/well.
Step 2 (Day 0 to Day 7): Induction of Intermediate Mesoderm Spheroid

The medium was changed to an intermediate mesoderm differentiation medium. As a basal medium for an intermediate mesoderm induction medium (KiO differentiation induction basal medium), APEL2 medium (STEMCELL Technologies) supplemented with 5% PFHM (Protein-Free Hybridoma Medium: Thermo Fisher) was used.
Step 2-1 (Day 0 to Day 3/4/5): In the first half of a period from Day 0 to Day 6, a medium obtained by adding CHIR99021 (CHIR) to the KiO differentiation induction basal medium was used as the intermediate mesoderm differentiation medium.
Step 2-2 (Day 3/4/5 to Day 6): In the second half of the period from Day 0 to Day 6, a medium obtained by adding 200 ng/ml FGF9 and 1 μg/ml heparin to the KiO differentiation induction basal medium was used as the intermediate mesoderm differentiation medium.

The medium was changed once every two days.

The numbers of days of the first half period (using CHIR) and the second half period (not using CHIR), and the concentration of CHIR added in the first half period were appropriately changed to make examinations described in Examples below.
Step 2-3 (Day 6 to Day 7): Formation of Intermediate Mesoderm Spheroid with 96-Well Plate

Cells on Day 6 of differentiation induction were dissociated into single cells with Accutase, and the resultant was suspended in a basal medium supplemented with 200 ng/ml FGF9 and 1 μg/ml heparin (KiO differential induction basal medium), and seeded in Prime Surface 96-well plate (Sumitomo Bakelite Co., Ltd.) at 5×10⁴ cells/well (medium: 200 μl/well). The resultant plate was centrifuged at 300 g for 3 minutes to precipitate cells, which were cultured overnight (for 1 day) in an incubator at 37° C. and 5% CO₂.

### (2) KiO Induction by Air-Liquid Interface Culture (ALIC)

Step 3 (Day 7): Air-Liquid Interface Culture with Transwell Plate

The intermediate mesoderm spheroid produced in Prime Surface 96-well plate was washed by transferring the intermediate mesoderm spheroid to a fresh plate of the same type (basal medium: 200 μl/well). After washing, the intermediate mesoderm spheroid was moved to an insert of a transwell plate. A basal medium supplemented with 5 μM CHIR was added to the respective wells of the transwell plate to form a liquid phase. The insert was inserted into the wells and allowed to stand still for 1 hour with the intermediate mesoderm spheroid held in the insert retained on the air-liquid interface in an incubator at 37° C. and 5% CO₂. Thereafter, the medium of the liquid phase was changed to a basal medium supplemented with 200 ng/ml FGF9 and 1 μg/ml heparin, and the resultant was cultured overnight. As the transwell plate, a 6-well transwell plate or a 24-well transwell plate was used.

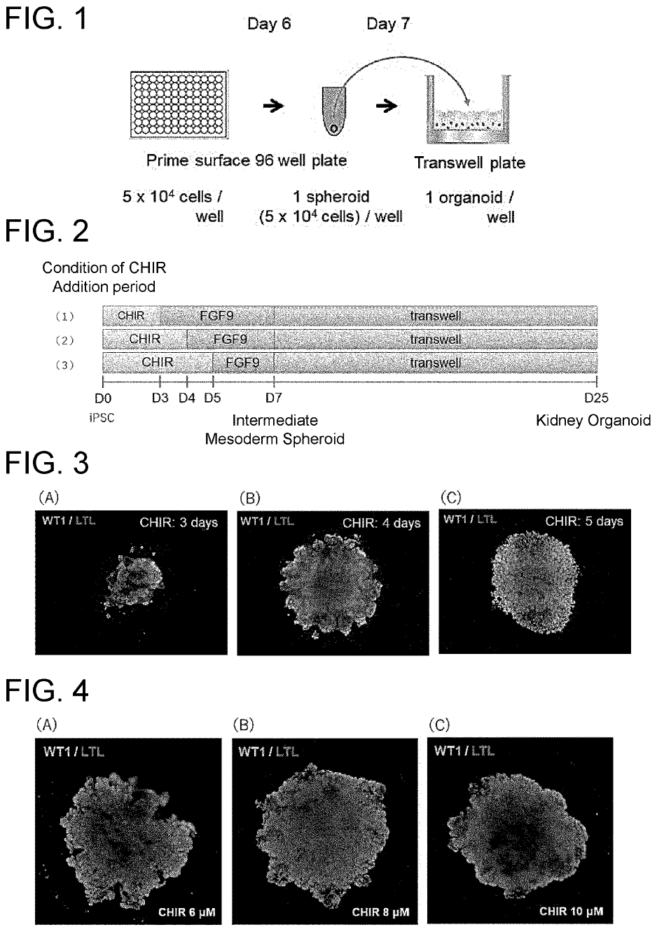

FIG. 1 illustrates the experimental scheme of the steps 2-3 and 3. When the present method is employed, a uniform intermediate mesoderm spheroid little varied in the type, the number, and the size of cells constituting the spheroid could be produced, and an operation for leaving the intermediate mesoderm spheroid to stand still on the air-liquid interface of the transwell plate could be stably performed.
Step 4 (Day 8 to Day 11): Culture with FGF9 and Heparin Supplemented

On Day 8 to Day 11, culture was performed with a basal medium supplemented with 200 ng/ml FGF9 and 1 μg/ml heparin added to the liquid phase. The medium was changed once every two days.
Step 5 (Day 12 to Day 25): Culture with Heparin Supplemented

On and after Day 12, culture was performed with a basal medium supplemented with 1 μg/ml of heparin added to the liquid phase. The medium was changed once every two days.

### Example 1: Examination of Influence of Addition Period and Concentration of CHIR on Ratio Between Glomerular Epithelial Cell-Like Cells and Renal Proximal Tubule Epithelial Cells in KiO

The addition period and the concentration of CHIR in the step 2 of the outline described above were changed to examine how the ratio between the glomerular epithelial cell-like cells and the renal proximal tubule epithelial cell-like cells changed.

As conditions of the addition period and concentration of CHIR, the following were examined:

### Conditions of Addition Period of CHIR (see FIG. 2)

Condition (1): the first half period (using CHIR): 3 days (Day 0 to Day 2)/the second half period (not using CHIR): 3 days (Day 3 to Day 5)
Condition (2): the first half period: 4 days (Day 0 to Day 3)/the second half period: 2 days (Day 4 to Day 5) or
Condition (3): the first half period: 5 days (Day 0 to Day 4)/the second half period: 1 day (Day 5)

### Conditions of Addition Concentration of CHIR

6 μM, 8 μM, and 10 μM.

The culture not using CHIR was performed in a medium obtained by supplementing a basal medium with 200 ng/ml FGF9 and 1 μg/ml heparin.

The step 1 (Day 1) and the steps 3 to 6 (Day 6 to Day 24) were performed in the same manner as in Example 1. As the air-liquid interface culture in the steps 4 to 6 (Day 7 to Day 24), however, a 6-well transwell plate made of PET was used.

FIG. 3 illustrates fluorescence microscope images of KiOs induced with the addition concentration of CHIR set to the same condition of 8 μM, and with the addition period of CHIR changed among the conditions (1), (2), and (3). When the condition (1) (CHIR added for 3 days) was employed, a KiO having a high ratio of the renal proximal tubule epithelial cell-like cells expressing LTL (red) of a renal proximal tubule marker was obtained (see FIG. 3(A)). On the other hand, when the condition (3) (CHIR added for 5 days) was employed, a KiO having a high ratio of the glomerular epithelial cell-like cells expressing WT1 (green) of a glomerular epithelial cell marker was obtained (see FIG. 3(C)). When the condition (2) (CHIR added for 4 days) was employed, a KiO having an intermediate ratio between the renal proximal tubule epithelial cell-like cells and the glomerular epithelial cell-like cells was obtained (see FIG. 3(B)).

FIG. 4 illustrates fluorescence microscope images of KiOs induced with the addition period of CHIR set to the condition (2) (CHIR added for 4 days), and with the addition concentration of CHIR changed among 6 μM, 8 μM, and 10 μM. When the addition concentration was 6 μM, a KiO having a high ratio of the renal proximal tubule epithelial cell-like cells expressing LTL (red) of the renal proximal tubule marker was obtained (see FIG. 4(A)). On the other hand, when the addition concentration was 10 μM, a KiO having a high ratio of the glomerular epithelial cell-like cells expressing WT1 (green) of the glomerular epithelial cell marker was obtained (see FIG. 4(C)). When the addition concentration was 8 μM, a KiO having an intermediate ratio between the renal proximal tubule epithelial cell-like cells and the glomerular epithelial cell-like cells was obtained (see FIG. 4(B)).

In the KiO obtained at the CHIR addition concentration of 6 μM, a large number of cells in the renal proximal tubule-like form were observed (see red broken lines of FIG. 5).

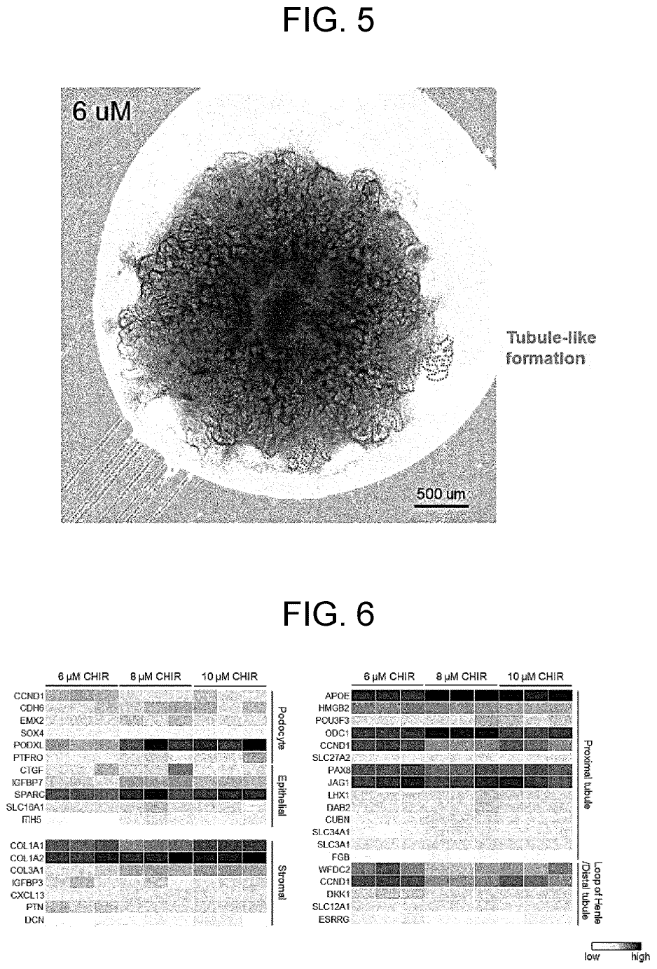

FIG. 6 illustrates gene expression patterns of the KiOs obtained at the CHIR addition concentrations of 6 μM, 8 μM, and 10 μM. When the addition concentration was 6 μM, expression of marker genes of renal proximal tubule epithelial cells (Proximal tubule) and loop of Henle/renal distal tubule (Loop of Henle/Distal tubule) tended to be high. On the other hand, when the addition concentration was 10 μM, expression of a marker gene of glomerular epithelial cell-like cells (Podocyte) tended to be high.

### Example 2: Air-Liquid Interface Culture With 24-Well Transwell Plate

It was examined whether or not a 24-well transwell plate could be used in the air-liquid interface culture performed in and after the step 3 (Day 7) of the outline described above.

The step 1 and the step 2 (Day 1 to Day 6) were performed in the same manner as described in the outline.

The intermediate mesoderm spheroid produced in Prime Surface 96-well plate was washed by transferring the intermediate mesoderm spheroid to a fresh plate of the same type (basal medium: 200 μl/well). After washing, the intermediate mesoderm spheroid was moved to an insert of a 24-well transwell plate (Kurabo Industries Ltd.) made of a PTFE (polytetrafluoroethylene) membrane material. A basal medium supplemented with 5 μM CHIR was added to the respective wells of the transwell plate to form a liquid phase (300 μl/well). The insert was inserted into the wells and allowed to stand still for 1 hour with the intermediate mesoderm spheroid held in the insert retained on the air-liquid interface in an incubator at 37° C. and 5% CO₂. Thereafter, the medium of the liquid phase was changed to a basal medium supplemented with 200 ng/ml FGF9 and 1 μg/ml heparin, and the resultant was cultured overnight.

The step 4 and the step 5 (Day 8 to Day 24) were performed in the same manner as described in the outline.

When the 24-well transwell plate was used, an operation for placing the intermediate mesoderm spheroid in the insert could be more stably performed than when the 6-well transwell plate was used. Besides, the KiO could be prepared stably without infiltration of the medium to the air phase. It was confirmed that the KiO prepared in the 24-well transwell plate had substantially the same gene expression patterns as the KiO prepared by the conventional method using the 6-well transwell plate (see Example 1 and Non Patent Literature 2) (no data).

### Example 3: Toxicity Evaluation Using KiO

### (1) Addition of Test Substance

A KiO (Day 25) produced on the air-liquid interface of a 24-well transwell plate in accordance with the method described in Example 2 was exposed to a test substance. Specifically, a basal medium (300 μl/well) supplemented with a solvent containing the test substance was introduced into the air phase of the transwell plate to replace the medium of the liquid phase with the basal medium supplemented with the test substance (500 μl/well). The resultant was cultured for 3 days in an incubator at 37° C. and 5% CO₂ to expose the KiO to the test substance.

### (2) Cytotoxicity Measurement 1: Measurement of Intracellular ATP Amount

The cytotoxicity of the test substance was measured based on the intracellular ATP amount.

Specifically, the KiO having been exposed to the test substance for 3 days was transferred from the transwell to a 1.5 ml tube, the resultant was centrifuged at 300 g for 5 minutes, and then, a supernatant was removed. The KiO was lysed in 100 μl of CellTiter-Glo 3D reagent (Promega), and further subjected to sonication to completely lyse the cells. The thus obtained cell lysate was transferred to a 96-well plate, and allowed to stand still under light shading at 25° C. for 25 minutes. A microplate reader was used to measure the amount of luminescence dependent on the ATP amount.

As a control substance, a solvent was singly used.

The cytotoxicity was calculated as “(measured value of test substance/measured value of control substance)×100%”.

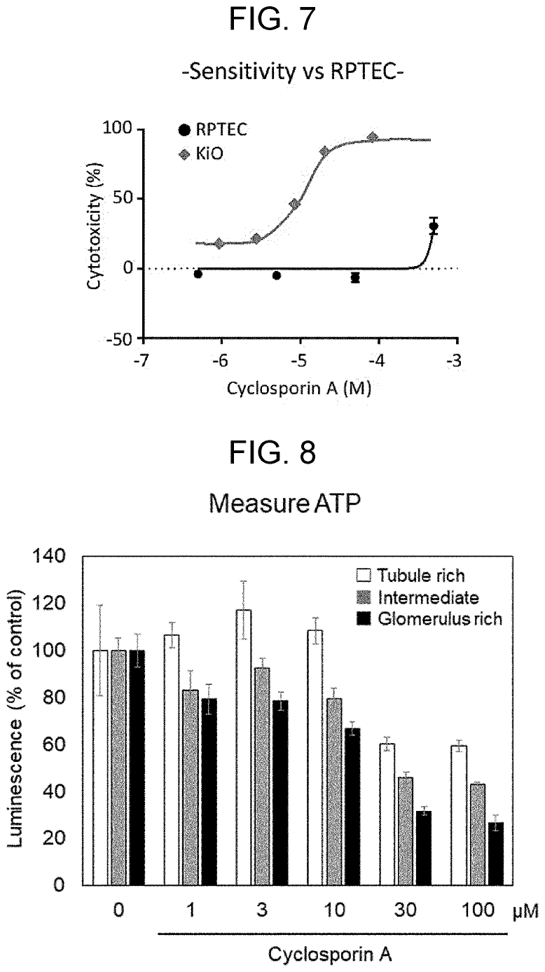

The results obtained by using Cyclosporin A (concentration: 1×10⁻⁷ M to 1×10⁻³ M) as the test substance are illustrated in FIG. 7. It is known that Cyclosporin A shows strong toxicity in the glomerulus. In a KiO produced in accordance with the method described in Example 3, cytotoxicity dependent on the concentration of Cyclosporin A could be detected. On the other hand, when the test was similarly performed by using, instead of the KiO, a renal proximal tubule epithelial cell line (RPTEC), the cytotoxicity dependent on the concentration of Cyclosporin A could not be detected.

Besides, in accordance with the method described in Example 1, a KiO having a high ratio of the renal proximal tubule epithelial cell-like cells, a KiO having a high ratio of the glomerular epithelial cell-like cells, and a KiO having an intermediate ratio between the renal proximal tubule epithelial cell-like cells and the glomerular epithelial cell-like cells were produced, and similarly subjected to the test. The results are illustrated in FIG. 8. In the KiO having a high ratio of the renal proximal tubule epithelial cell-like cells (Tubule rich), higher cytotoxicity was detected, and in the KiO having a high ratio of the glomerular epithelial cell-like cells (Glomerulus rich), lower cytotoxicity could be detected. When the KiOs different in the ratio between the renal proximal tubule epithelial cell-like cells and the glomerular epithelial cell-like cells produced in accordance with the method described in Example 1 were used, a cytotoxicity profile of Cyclosporin A (strong cytotoxicity in the glomerulus) could be thus detected.

### (3) Cytotoxicity Measurement 2: Measurement of LDH Leakage Amount

The cytotoxicity of a test substance was measured based on the amount of LDH leaked from cells.

Specifically, after exposing a KiO produced in accordance with the method described in Example 1 to the test substance for 3 days, and then, 100 μl of the medium (liquid phase) was collected in a 96-well plate. The resultant was mixed with 100 μl of a LDH measurement buffer (Cytotoxicity LDH Assay Kit-WST: DOJINDO LABORATORIES), and the resultant mixture was allowed to stand still for 30 minutes in an incubator at 37° C. and 5% CO₂. 50 μl of a stop solution was added thereto to stop the reaction, and an absorbance at 490 nm was measured with a microplate reader.

As a control substance, a solvent was singly used.

The cytotoxicity was calculated as “(measured value of test substance/measured value of control substance)×100%”.

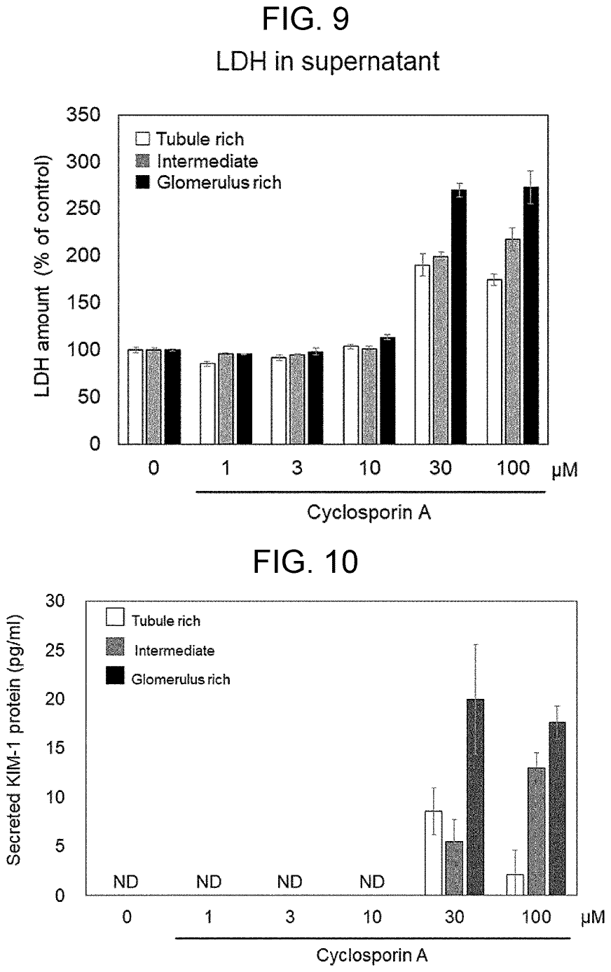

The results are illustrated in FIG. 9. In all the KiOs, cytotoxicity dependent on the concentration of Cyclosporin A was observed. Besides, when the KiO having a high ratio of the glomerular epithelial cell-like cells was used (Glomerulus rich), toxicity was easily observed.

### (4) Cytotoxicity Measurement 3: Measurement of Secreted Marker Protein

The cytotoxicity of a test substance was measured based on the amount of nephrotoxicity marker KIM-1 (Kidney Injury Marker 1) secreted into the medium.

KIM-1 was quantitatively determined with KIM-1 ELISA kit (R & D). A KiO produced in accordance with the method described in Example 1 was exposed to the test substance for 3 days, and then, 50 μl of the medium (liquid phase) was collected and transferred to a 96-well plate in which an anti-KIM-1 capture antibody was immobilized. The resultant was allowed to stand still under light shading at 25° C. for 2 hours. The resultant wells were washed with a cleaning solution containing a surfactant four times, and 200 μl of a buffer containing an anti-KIM-1 detection antibody was added thereto. The resultant was allowed to stand still under light shading at 25° C. for 2 hours. The resultant wells were washed with a cleaning solution containing a surfactant four times, and 200 μl of a substrate solution was added thereto. The resultant was allowed to stand still under light shading at 25° C. for 30 minutes. The reaction was stopped by adding 50 μl of a stop solution, and an absorbance at 450 nm was measured with a microplate reader.

As a control substance, a solvent was singly used.

The cytotoxicity was calculated as “(measured value of test substance/measured value of control substance)×100%”.

The results are illustrated in FIG. 10. Also when the amount of secreted KIM-1 was used as the indicator, the cytotoxicity of Cyclosporin A could be detected.

### Example 4: Improved Method for Differentiation Induction of iPSC-Derived KiO

(1) Differentiation Induction From iPSC Into Intermediate Mesoderm Spheroid
Step 1 (Day 1): iPSC Seeding

The seeding was performed by the same method as described above in the outline.
Step 2 (Day 0 to Day 5): Induction of Intermediate Mesoderm Spheroid

In the method described above in the outline, the period of the step 2-1 (first half period) was set to from Day 0 to Day 4, and the period of the step 2-2 (second half period) was set to from Day 4 to Day 5.

The medium was changed to an intermediate mesoderm differentiation medium. As a basal medium of an intermediate mesoderm induction medium (KiO differentiation induction basal medium), a medium obtained by adding 5% PFHM (Protein-Free Hybridoma Medium: Thermo fisher) to APEL2 medium (STEMCELL Technologies) was used.
Step 2-1 (Day 0 to Day 4): In the first half of the period from Day 0 to Day 5, a medium obtained by adding CHIR99021 (CHIR) to the KiO differentiation induction basal medium was used as the intermediate mesoderm differentiation medium.
Step 2-2 (Day 4 to Day 5): In the second half of the period from Day 0 to Day 5, a medium obtained by adding 200 ng/ml FGF9 and 1 μg/ml heparin to the KiO differentiation induction basal medium was used as the intermediate mesoderm differentiation medium.

The medium was changed once every two days.
Step 2-3 (Day 5 to Day 7): Formation of Intermediate Mesoderm Spheroid with 96-Well Plate

The period from Day 6 to Day 7 of the step 2-3 of the method described above in the outline was changed to a period from Day 5 to Day 7 in this improved method.

Cells on Day 5 of differentiation induction were dissociated into single cells with Accutase, and the resultant was suspended in a basal medium supplemented with 200 ng/ml FGF9 and 1 μg/ml heparin (KiO differential induction basal medium), and seeded in Prime Surface 96-well plate (Sumitomo Bakelite Co., Ltd.) at 5×10⁴ cells/well (medium: 200 μl/well). The resultant plate was centrifuged at 300 g for 3 minutes to precipitate the cells, which were cultured for 2 days in an incubator at 37° C. and 5% CO₂.

Since the culture period was increased from 1 day to 2 days, a spheroid solid with cells at the center (having a clear core) could be formed, and a KiO was more stably induced in the subsequent step 3.

### (2) KiO Induction by Air-Liquid Interface Culture (ALIC)

Step 3 (Day 7): Air-Liquid Interface Culture with Transwell Plate

The culture was performed in the same manner as in the method described above in the outline except that a membrane of an insert of a transwell plate was precedently hydrophilized.

The intermediate mesoderm spheroid produced in Prime Surface 96-well plate was washed by transferring the intermediate mesoderm spheroid to a fresh plate of the same type (basal medium: 200 μl/well). After washing, the intermediate mesoderm spheroid was moved to an insert of a transwell plate. Two days before the use, the insert of the transwell plate was immersed in water (upper layer: 500 μl, lower layer: 1 ml) for hydrophilizing the membrane. An insert poor in hydrophilicity was excluded from the use. A basal medium supplemented with 5 μM CHIR was added to the respective wells of the transwell plate to form a liquid phase. The insert was inserted into the wells and allowed to stand still for 1 hour with the intermediate mesoderm spheroid held in the insert retained on the air-liquid interface in an incubator at 37° C. and 5% CO₂. Thereafter, the medium of the liquid phase was changed to a basal medium supplemented with 200 ng/ml FGF9 and 1 μg/ml heparin, and the resultant was cultured overnight.

Since the membrane was hydrophilized, extension of the spheroid on the membrane is accelerated, and thus, a flatter spheroid with a smaller height could be formed in the insert. In this manner, necrosis of cells present in a higher position in the spheroid (a position farther from the membrane surface) otherwise caused because nutrient supply from the air-liquid interface was insufficient could be suppressed.
Step 4 (Day 8 to Day 11): Culture with FGF9 and Heparin Supplemented
Step 5 (Day 12 to Day 25): Culture with Heparin Supplemented

These steps were performed in the same manner as in the method described above in the outline except that an operation for washing necrotic cells away was performed.

From Day 8 to Day 11, the culture was performed with a basal medium supplemented with 200 ng/ml FGF9 and 1 μg/ml heparin added to the liquid phase. The medium was changed once every two days. On and after Day 12, the culture was performed with a basal medium supplemented with 1 μg/ml heparin added to the liquid phase. The medium was changed once every two days.

Once a week, water was dropped onto the KiO from above to wash necrotic cells away. In this manner, the viability of cells included in the KiO was improved, and the KiO was improved also in the visibility.

1. A method for evaluating nephrotoxicity of a test substance comprising:
a step of contacting a test substance with a kidney organoid,
wherein the higher cytotoxicity of test substance as compared with a control substance indicates the test substance is likely to have nephrotoxicity.

2. A method for screening a test substance likely to have nephrotoxicity comprising the steps of:
(1) contacting a test substance with a kidney organoid; and
(2) selecting a test substance showing higher cytotoxicity as compared with a control substance.

3. The method according to claim 1, wherein the kidney organoid is derived from a human induced pluripotent stem cell.

4. The method according to claim 1, wherein the kidney organoid is enriched in glomerular epithelial cell-like cells.

5. The method according to claim 4, wherein 30 to 80% of cells contained in the kidney organoid are glomerular epithelial cell-like cells.

6. The method according to claim 4 further comprising:
a step (a) of culturing a pluripotent stem cell in the presence of a 9.0 to 10.5 μM GSK3β inhibitor to obtain the kidney organoid enriched in glomerular epithelial cell-like cells.

7. The method according to claim 1, wherein the kidney organoid is enriched in renal proximal tubule epithelial cell-like cells.

8. The method according to claim 7, wherein 5 to 30% of cells contained in the kidney organoid are renal proximal tubule epithelial cell-like cells.

9. The method according to claim 8 further comprising:
a step (b) of culturing a pluripotent stem cell in the presence of a 5.5 to 8.5 μM GSK3β inhibitor to obtain the kidney organoid enriched in renal proximal tubule epithelial cell-like cells.

10. A method for determining a site in the kidney to which a test substance shows toxicity comprising the steps of:
(A) contacting a test substance with a kidney organoid enriched in glomerular epithelial cell-like cells;
(B) contacting the test substance with a kidney organoid enriched in renal proximal tubule epithelial cell-like cells; and
(C) comparing cytotoxicity obtained in the step (A) and cytotoxicity obtained in the step (B) to determine that a site showing toxicity is the glomerulus when the cytotoxicity obtained in the step (A) is higher, and that a site showing toxicity is the renal proximal tubule when the cytotoxicity obtained in the step (B) is higher.

11. A method for screening nephrotoxicity of a drug candidate compound comprising the steps of:
(1) contacting a test substance with a kidney organoid enriched in glomerular epithelial cell-like cells; and
(2) selecting, as the drug candidate compound, a test substance showing no cytotoxicity or lower cytotoxicity as compared with a control substance.

12. A kit for evaluating nephrotoxicity of a test substance comprising a kidney organoid.

13. A method for producing a kidney organoid enriched in glomerular epithelial cell-like cells comprising:
a step (a) of culturing a pluripotent stem cell in the presence of a 9.0 to 10.5 μM GSK3β inhibitor.

14. A method for producing a kidney organoid enriched in renal proximal tubule epithelial cell-like cells comprising:
a step (b) of culturing a pluripotent stem cell in the presence of a 5.5 to 8.5 μM GSK3β inhibitor.
